(19) United States
(12) Patent Application Publication
(10) Pub. No.: US 2008/0283553 A1
(43) Pub. Date: Nov. 20, 2008

(54) Closure for a Dispensing Device

(75) Inventors: Mark Anthony Cox, Hertfordshire (GB); Paul Kenneth Rand, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Middlesex, EN (GB)

(21) Appl. No.: 12/066,667

(22) PCT Filed: Sep. 12, 2006
(86) PCT No.: PCT/EP2006/008987

§ 371 Date: Mar. 13, 2008

(30) Foreign Application Priority Data
Sep. 14, 2005 (GB) 0518770.3

(51) Int. Cl. B67B 5/00; A61M 11/00

(52) U.S. Cl. 222/153.11; 128/200.23; 222/153.13

(57) ABSTRACT

A closure is provided for use with a dispensing device which comprises a housing having a dispensing outlet, a supply of a substance and a dispensing member mountable in the housing in a rest position relative to the housing and movable from the rest position to an actuated position, said movement from the rest position to the actuated position causing dispensing of said substance out of the dispensing outlet. The closure is adapted to close the dispensing outlet in two different orientations of the closure. Moreover, the closure has a restricting member which, when the closure closes the dispensing outlet in either orientation, prevents movement of the dispensing member from the rest position to the actuated position so that dispensing of the substance is prevented when the closure closes the dispensing outlet.

### FIELD OF THE INVENTION

The present invention relates to a closure for a dispensing device, and is particularly, but not exclusively, concerned with a closure for a medicament dispenser.

An example of a medicament dispenser to which the invention is particularly, but not exclusively, concerned is an inhaler, for instance a pressurised metered dose inhaler (hereinafter referred to as a “pMDI”). The invention does, however, embrace other inhaler types, for example a dry powder inhaler (DPI), as will be appreciated by the reader skilled in the inhaler art.

### BACKGROUND OF THE INVENTION

pMDIs are well known in the art of inhalation devices. It is therefore not necessary to describe the construction and operation of a pMDI other than in bare essentials.

A pMDI comprises a canister unit and a housing. The housing is generally tubular, although this is not essential, and generally formed of a plastics material, for instance by moulding. The canister unit comprises an open-ended canister, typically made from a metal such as aluminium. The open end of the canister is sealingly capped by a metering valve assembly. The valve assembly includes a hollow dispensing member or valve stem which projects from the outlet or business end of the canister. The dispensing member is mounted for sliding movement relative to the canister between an extended position, to which the dispensing member is biased by a biasing mechanism in the valve assembly, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S.A.S. (Le Neubourg, France).

The housing comprises an internal passageway having an open end. The canister unit is slidable into the internal passageway through the open end with the canister unit being inserted valve assembly first into the internal passageway. A stem block, which receives the dispensing member of the canister when the canister unit is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister unit from its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister unit from the rest position to the actuated position. The inspiratory airflow produced by the patient entrains the metered dose of the medicinal aerosol formulation into the patient's respiratory tract.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 and WO-A-2004/001664 to Glaxo Group Limited, incorporated herein by reference. The pMDI canister unit may comprise the dose counter, which is fixably secured on the valve assembly end of the canister and includes a display which denotes the number of metered doses of the medicament formulation dispensed from, or remaining in, the canister. The display of the dose counter is visible to the patient through a window provided in the housing. The display may be presented by a plurality of indicator wheels rotatably mounted on a common axle, each wheel having numerals from ‘0’ to ‘9’ displayed in series around the circumference.

pMDI devices, however, are susceptible to unintentional actuation, particularly whilst in transit, for example shipment between the manufacturer and distributor. During such transit, such devices and their packaging are often subjected to impacts and sudden movements. Such forces can actuate the pMDI, causing doses of the formulation to be dispensed. When the pMDI includes a dose counter, rough handling in transit can cause the value displayed to the user by the counter to increase or decrease so that it is not consistent with the number of doses that have been dispensed by, or remain in, the pMDI. It is wasteful to dispense unwanted doses of the medicament, and potentially very dangerous for a dose counter to indicate to the user that more doses remain in the canister than are actually present.

It is therefore desirable to provide a pMDI that is adapted to prevent unintentional actuation. It is also desirable to provide a pMDI with a dose counter which is adapted to prevent miscounting actuations in the event of an impact.

In International patent application No. PCT/GB2005/000926 (publication No. WO-A-2005/087299) there is disclosed a dust cap for a pMDI which is provided with a restricting member which passes through the dispensing outlet when the dust cap covers the dispensing outlet. The restricting member acts to restrict movement of the canister unit in the housing whereby actuation of the pMDI is prevented, as is actuation of the dose counter. For the restricting member to operate correctly, the dust cap has to be applied to the dispensing outlet in a correct angular orientation. However, the dust cap has a body which is complementary to that of the dispensing outlet in two different angular orientations, only one of which is the correct orientation. There is therefore a possibility that a patient will attempt to apply the dust cap to the dispensing outlet in the incorrect orientation and which, if left, will prevent the restricting member from stopping inadvertent operation of the pMDI. As an example, depression of the canister unit in the housing may result in the dust cap being detached from the dispensing outlet due to the canister unit applying a camming force on the restricting member.

In consideration of this problem, International patent application No. PCT/GB2005/000926 further discloses dust caps which are adapted so that, if a patient attempts to apply the dust cap to the dispensing outlet in the wrong angular orientation, it will be clear that this is the case to the patient.

It would be useful if the dust cap could be applied in either orientation and, irrespective of the orientation, the restricting member was still able to restrict movement of the canister unit in the housing to prevent inadvertent actuation of the canister unit.

In at least one aspect of the present invention there is provided a closure for a dispensing device which meets this aim.

### SUMMARY OF THE INVENTION

According to one aspect of the present invention there is provided a closure as set forth in claim 1 hereof.

According to a second aspect of the present invention there is provided a closure as set forth in claim 2 hereof.

Additional aspects and features of the present invention are set forth in the subsidiary claims and in the description of an exemplary embodiment of the present invention which now follows with reference to the accompanying Figures of drawings.

### BRIEF DESCRIPTION OF THE DRAWINGS

FIG. 1 is a perspective view of a pMDI comprising a housing in which a canister unit is slidably mounted.

FIG. 2 is a schematic, part scrap, view of the pMDI.

FIG. 3 is a schematic, perspective view of a closure for mounting on a mouthpiece of the pMDI housing.

FIG. 4 is a plan view of the closure.

FIG. 5 is a schematic, fragmentary side elevation of the pMDI with the closure mounted to the mouthpiece of the pMDI housing.

### DETAILED DESCRIPTION OF THE DRAWINGS

The FIGURES show a hand-held, hand-operable pMDI according to an embodiment of the present invention. In this embodiment, the pMDI is based on a pMDI known in the prior art, as described in the ‘Background of the Invention’ section supra, although the present invention is not limited to the exact form of such an arrangement.

The pMDI comprises a canister unit 14 and a housing 1 in which the canister unit 14 is slidable along its longitudinal axis L-L. The housing 1 is generally tubular and of L-shape having an axial section 1a and a transverse section 1b configured as a mouthpiece 3. The housing 1 is preferably moulded from a plastics material, for example by injection moulding. Conveniently, the housing is of polypropylene.

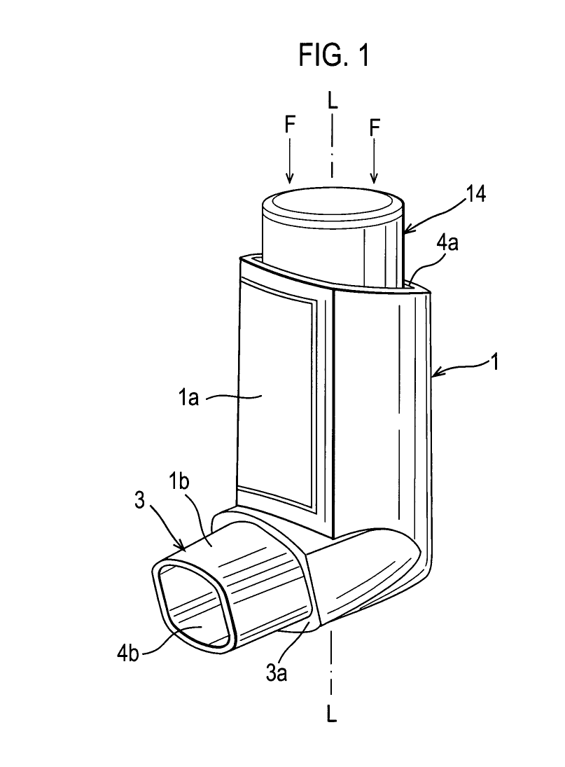

In the use orientation of the pMDI shown in FIG. 1, the housing 1 has an upper open end 4a in the axial section 1a, through which the canister unit 14 is reversibly slidable into the housing 1, and a lower open end 4b in the mouthpiece 3.

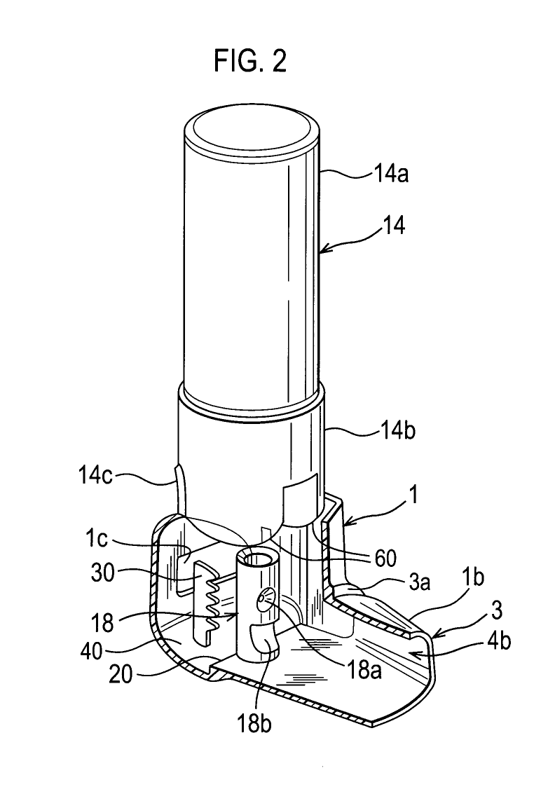

As shown in FIG. 2, the canister unit 14 comprises a pressurised canister 14a having a metering valve (not shown) at its leading or business end and a dose counter module 14b permanently mounted on the leading (valve) end of the canister 14a. The metering valve may be of the form set forth in U.S. Pat. Nos. 6,170,717; 6,315,173; and 6,318,603. The dose counter module 14b is as described and shown in WO-A-2004/001664 supra.

The canister 14a is made from metal, for instance aluminium, and may have its inner surface coated with a fluorocarbon polymer, optionally in a blend with a non-fluorocarbon polymer, such as a blend of polytetrafluoroethylene and polyethersulphone (PTFE-PES), as disclosed in U.S. Pat. Nos. 6,143,277; 6,511,653; 6,253,762; 6,532,955; and 6,546,928.

The canister 14a contains a pressurised medicinal aerosol formulation, as known in the art and mentioned briefly hereinabove. The formulation may be a suspension or solution formulation incorporating a propellant, typically a CFC-free propellant such as HFA-134a and/or HFA-227.

As further shown in FIG. 2 the housing 1 has an internal base surface 40 in which a step 20 is formed. The base surface 40 also supports an upwardly projecting stem block 18 which, as known in the art, receives a valve stem (not shown) of the metering valve. As further shown, the stem block 18 has a spray orifice 18a oriented towards the lower open end 4b in the mouthpiece 3 whereby the metered dose fired from the canister unit 14 on depression thereof into the housing 1 is directed out of the mouthpiece 3 for inhalation by the patient, as known in the art.

The dose counter module 14b has a display window 14c which displays the number of metered doses of the medicament formulation left in the canister 14a, as described in WO-A-2004/001664 supra. The housing 1 has a cut-out or window 1c through which the patient can see the dose counter display 14c.

As detailed in WO-A-2004/001664, the dose counter module 14b has a counting mechanism which is driven through a rack-and-pinion mechanism. FIG. 2 shows the rack 30 which also projects upwardly from the housing base surface 40. The rack is slidingly received in an aperture (not shown) in the leading face of the dose counter module 14b. When the canister unit 14 is depressed into the housing 1 for opening of the metering valve, the rack drives a pinion (not shown) in the dose counter module 14b and a rotary movement of the pinion causes the counting mechanism to decrement the number displayed in the dose counter window 14c by dose counter wheels (not shown).

In use, a patient in need of a metered dose of the medicinal aerosol formulation places his or her lips on the mouthpiece 3 of the housing 1 and then concurrently inhales and, with their finger(s), depresses the canister unit 14 into the housing 1 (arrows F, FIG. 1) to cause the metering valve to release a metered dose of the medicinal formulation from the canister unit 14 for entrainment in the inspiratory airflow produced by the patient for deposition in their lungs. The depression of the canister unit 14 into the housing 1 also results in the dose counter module 14b recording the release of the dose and showing the remaining number of metered doses left in the canister 14a.

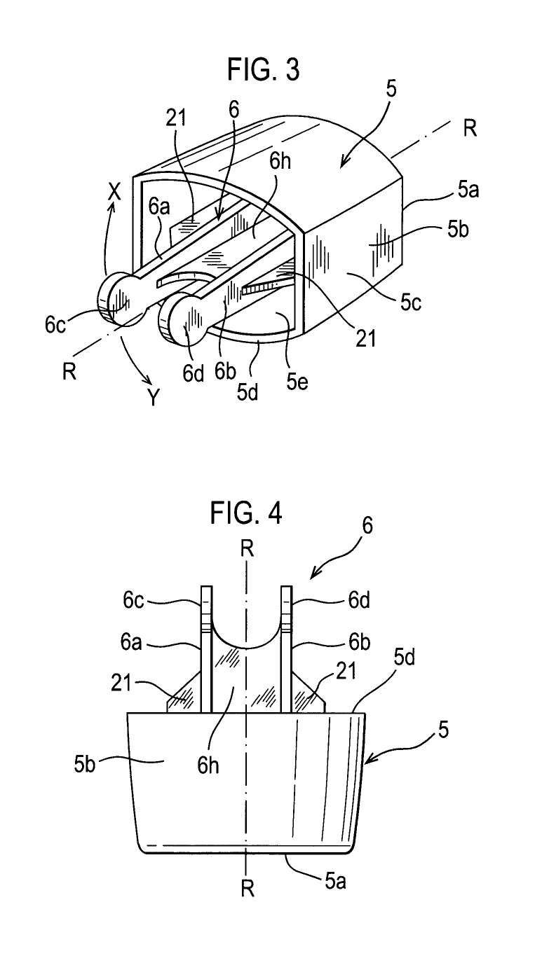

Referring to FIGS. 3 to 5, the pMDI further has a dust cap 5 for detachably engaging the mouthpiece 3. The dust cap 5 has a hollow body 5b which is of a shell form and a generally rectangular cross-sectional shape. The body 5b has a front face 5a and a side skirt 5c extending rearwardly from the front face 5a. The rear end of the side skirt 5c presents an annular lip 5d about a mouth 5e to the inner volume of the body 5b. The dust cap 5 may be moulded from polypropylene (PP), although, of course, other materials, in particular plastics materials, and forming techniques, may be used.

The dust cap 5 includes a restricting member 6 in the form of an arm or prong structure comprising a pair of spaced apart arms 6a, 6b. The arms 6a, 6b of the restricting member 6 are interconnected along part of their length by a strengthening rib 6h, in order to increase their strength and rigidity. Moreover, the restricting member 6 is provided with:—
(i) a clip 6c, 6d at the free end of each arm 6a, 6b for clipping to the step 20 in the housing 1, and
(ii) a pair of lateral alignment ribs (wings) 21,
   as will be described in further detail hereinafter. The clips 6c, 6d are formed by providing the free ends of the arms 6a, 6b as a ‘lollipop’ profile.

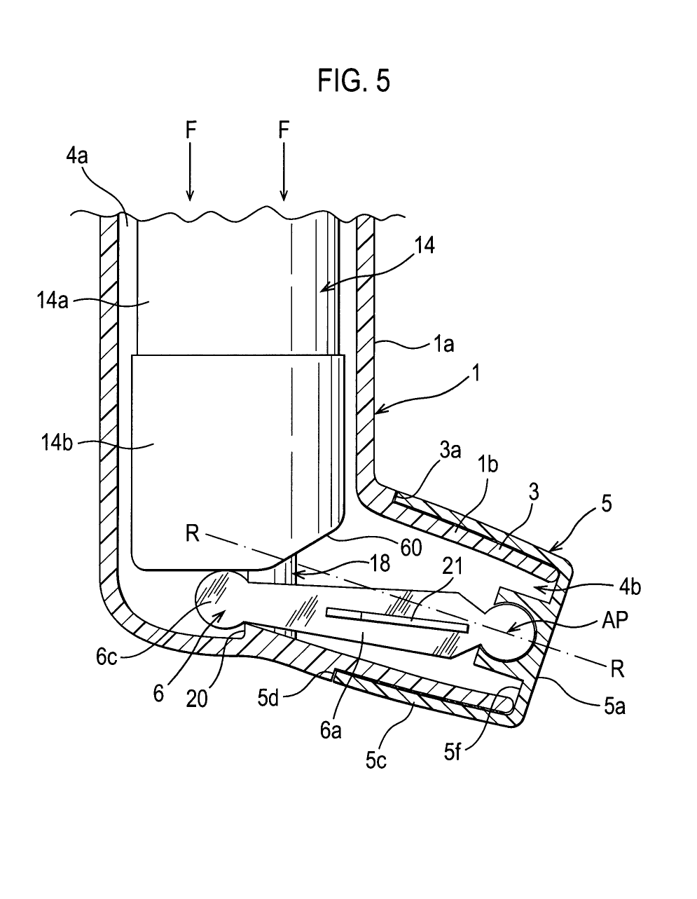

The restricting member 6 is pivotally attached at an attachment point AP (see FIG. 5) on an inner surface 5f of the front face 5a of the cap body 5b and extends rearwardly from the attachment point AP so as to protrude from the mouth 5e of the cap body 5b. The attachment point AP is symmetrically located in the cap 5 on a central axis R—R of the cap 5. The restricting member 6 is pivotable about the attachment point AP between a first pivot position, in which the restricting member 6 is angled away from the attachment point AP to one side of the central axis R—R in the direction of arrow X (see FIG. 3), and a second pivot position, in which the restricting member is angled away from the attachment point AP to another, opposed side of the central axis R—R in the direction of arrow Y (see FIG. 3). Typically, the restricting member 6 is free to pivot in the range of 15 to 30 degrees about the attachment point AP, that is to say, in the range of 7.5 to 15 degrees to either side of the central axis-R. Of course, the degree of pivotal freedom could be made different if desired.

In this particular embodiment of the invention, the cap body 5b and the restricting member 6 are formed as separate component parts of the cap 5. As will be understood by recourse to FIG. 5, the proximal end of the restricting member 6 is then assembled to the cap body 5b by a snap-fit connection of the proximal end to the attachment point AP. Of course, other suitable assembly connections could be used and are within the scope of the present invention. Moreover, in another embodiment the cap 5 may be integrally moulded with the restricting member 6, for instance a living hinge at the attachment point providing the pivot function.

As will be described in more detail hereinafter, when the dust cap 5 is mounted on the mouthpiece 3 the restricting member 6 prevents the canister unit 14 being depressed sufficiently far in the housing 1 to either (a) cause the dose counter module 14b to record a dose release event, or (b) cause the metering valve to open for release of a metered dose of the medicament formulation. The restricting member 6 thus prevents inadvertent counting and firing when the dust cap 5 is mounted on the mouthpiece 3, which is nearly all of the time as the dust cap 5 is only removed from the mouthpiece 3 when the patient needs a dose of the medicament formulation. Such inadvertent counting and firing might occur, for example, in the absence of the restricting member 6, during shipping of the pMDI from the manufacturer to the distributor, or when the pMDI is in a patient's pocket or handbag, or even as a result of a person fiddling/playing with the pMDI.

The mouthpiece 3 of the pMDI housing 1 is of complementary shape and size to the cap body 5b such that the cap body 5b is slidable rectilinearly over the mouthpiece 3 as a push-fit in two, opposing orientations of the cap 5 about its central axis R—R, i.e. orientations which differ by 180 degrees. It will also be appreciated that the mutual shapes of the cap body 5b and the mouthpiece 3 ensure that the cap 5 is non-rotatable on the mouthpiece 3 in either of the two cap mounting orientations. Moreover, in each cap mounting orientation the annular lip 5d of the side skirt 5c abuts an annular surface 3a of the pMDI housing 1 about the mouthpiece 3 so that there is no gap therebetween.

When the dust cap 5 is positioned on the mouthpiece 3 in either of the two cap orientations, the restricting member 6 extends into the housing 1 through the mouthpiece 3 such that the arms 6a, 6b straddle the stem block 18 to sit underneath the dose counter module 14b at the leading end of the canister unit 14. In this regard, the lateral alignment ribs 21 prevent the restricting member 6 from being inserted at more than a prescribed angle to the mouthpiece 3 to prevent or inhibit one of the arms 6a, 6b being inserted into a hollow 18b in the stem block 18 or being otherwise obstructed by the components of the pMDI. In other words, the alignment ribs 21 help to ensure that the dust cap 5 is mounted on the mouthpiece 3 so that the arms 6a, 6b straddle the stem block 18.

The pivotal nature of the restricting member 6 means that the restricting member 6 is locatable underneath the dose counter module 14b irrespective of which of the two cap orientations the cap 5 is slid onto the mouthpiece 3 in. The restricting member 6 will either be in the correct pivot position to pass underneath the dose counter module 14b or, if not, as would be the case where the cap 5 is slid onto the mouthpiece 3 in a different orientation from that in which it was removed, then the restricting member 6 is pivoted to a position in which it is located underneath the dose counter module 14b by engagement of the restricting member 6 with a deflecting surface 60 of the dose counter module 14b as the cap 5 is slid onto the mouthpiece 3. Either way, the restricting member 6 ends up located underneath the dose counter module 14b.

With the restricting member 6 disposed underneath the dose counter module 14b, the clips 6c, 6d are free to clip to the step 20 in the housing 1. In this regard, the clips 6c, 6d may automatically clip to the step 20 as the restricting member 6 slides underneath the dose counter module 14b. Otherwise, the clips 6c, 6d are brought into clipping engagement with the step 20 by the first (inadvertent) downward movement of the canister unit 14 in the housing 1 after mounting of the cap 5 on the mouthpiece 3 causing the restricting member 6 to pivot downwardly and the clips 6c, 6d to engage the step 20.

With the cap 5 mounted to the mouthpiece 3 in either of its two cap mounting orientations, and the restricting member 6 disposed underneath the leading end of the dose counter module 14b, inadvertent operation of the pMDI is prevented because the restricting member 6 blocks downward movement of the canister unit 14 in the housing 1 far enough for the metering valve to open, and hence a metered dose of the medicament formulation to be dispensed through the valve stem into the stem block 18 and sprayed from the spray orifice 18a towards the mouthpiece 3. Additionally, the restricting member 6 blocks the canister unit 14 from travelling far enough downwards in the housing 1 for operation of the dose counter module 14b.

Moreover, if the canister unit 14 is moved downwardly in the housing 1 while the cap 5 is mounted on the mouthpiece 3, the leading end of the canister unit 14 will push down on the arms 6a, 6b which results in the clips 6c, 6d engaging the step 20 more firmly thereby ensuring that the cap 5 is not ejected from the mouthpiece 3 and that inadvertent counting and firing is prevented.

By having the restricting member 6 extend through the mouthpiece 3, no changes need to be made to the housing 1 to accommodate it. Thus, the dust cap 5 can be used with an existing pMDI housing. Moreover, the profile of the inhalation airflow through the housing 1, which flows into the housing 1 through the upper open end 4a and out of the housing 1 through the lower open end 4b, is unaffected by the provision of the restricting member 6, since it requires no change to the housing 1 and is removed from the housing 1 prior to use of the pMDI. Consequently, the pharmaceutical performance of the pMDI is unaffected by the provision of the restricting member 6 avoiding the need to obtain new regulatory approval for an existing pMDI product using the new dust cap 5. Nonetheless, the dust cap 5 could be used with a new housing, if desired.

The medicament contained in the canister unit 14 may for the treatment of mild, moderate or severe acute or chronic symptoms or for prophylactic treatment. The medicament is suitably for treating respiratory diseases, e.g. asthma, chronic obstructive pulmonary disease (COPD), although may be for other therapeutic indications, e.g. treating rhinitis.

Appropriate therapeutic agents or medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., becomehtasone (e.g. as the dipropionate ester), fluticasone (e.g. as the propionate ester), flunisolide, budesonide, rofleponide, mometasone (e.g. as the furoate ester), ciclesonide, triamcinolone (e.g. as the acetonide), 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester or 6α, 9α-Difluoro-17α-[(2-furanylcarbonyl)oxy]-11β-hydroxy-16α-methyl-3-oxo-androsta-1,4-diene-17β-carbothioic acid S-fluoromethyl ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H) benzo-thiazolone; PDE4 inhibitors e.g. cilomilast or roflumilast; leukotriene antagonists e.g. montelukast, pranlukast and zafirlukast; [adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate)]; [α4 integrin inhibitors e.g. (2S)-3-[4-({[4-(aminocarbonyl)-1-piperidinyl]carbonyl}oxy)phenyl]-2-[((2S)-4-methyl-2-{[2-(2-ethylphenoxy)acetyl]amino}pentanoyl)amino]propanoic acid (e.g as free acid or potassium salt)], diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagons. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament and/or to minimise the solubility of the medicament in the propellant.

Preferably, the medicament is an anti-inflammatory compound for the treatment of inflammatory disorders or diseases such as asthma and rhinitis.

Preferably, the medicament is formulated in a hydrofluoroalkane propellant, such as HFA-134a or HFA-227 or a combination thereof.

Preferably, the medicament is an anti-inflammatory steroid, such as a corticosteroid, for instance fluticasone, e.g. as the propionate ester, or a long acting beta agonist (LABA), such as salmeterol, e.g. as the xinafoate salt, or a combination thereof.

Preferred medicaments are salmeterol, salbutamol, albuterol, fluticasone and beclomethasone and salts, esters or solvates thereof, for instance fluticasone propionate, albuterol sulphate, salmeterol xinafoate and beclomethasone dipropionate.

The medicament may also be a glucocorticoid compound, which has anti-inflammatory properties. One suitable glucocorticoid compound has the chemical name: 6α, 9α-Difluoro-17α-(1-oxopropoxy)-11β-hydroxy-16α-methyl-3-oxo-androsta-1,4-diene-17β-carbothioic acid S-fluoromethyl ester (fluticasone propionate). Another suitable glucocorticoid compound has the chemical name: 6α, 9α-difluoro-17α-[(2-furanylcarbonyl)oxy]-11β-hydroxy-16α-methyl-3-oxo-androsta-1,4-diene-17β-carbothioic acid S-fluoromethyl ester. A further suitable glucocorticoid compound has the chemical name: 6α, 9α-Difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

Other suitable anti-inflammatory compounds include NSAIDs e.g. PDE4 inhibitors, leukotriene antagonists, INOS inhibitors, tryptase and elastase inhibitors, beta-2 integrin antagonists and adenosine 2a agonists.

The medicaments may be delivered in combinations. As an example, there may be provided salbutamol (e.g. as the free base of the sulphate salt) or salmeterol (e.g. as the xinafoate salt) in combination with an anti-inflammatory steroid, such as beclomethasone (e.g. as an ester, preferably dipropionate) or fluticasone (e.g. as an ester, preferably propionate).

For the avoidance of doubt, it will be appreciated that the present invention is equally applicable for a pMDI having a canister unit which does not include a dose counter module. That is to say, the canister unit may simply be a pressurised canister and the restricting member interacts with the canister to prevent inadvertent downward movement thereof. Alternatively, some other accessory or cap or module may be mounted to the leading end of the canister in place of the dose counter module for the restricting member to interact with.

All publications, patents, and patent applications cited herein are hereby incorporated herein by reference in their entirety to the same extent as if each publication, patent, or patent application was specifically and individually indicated to be incorporated by reference.

It must be noted that, as used in the specification and appended claims, the singular forms “a”, “an”, “the” and “one” include plural referents unless the content clearly dictates otherwise.

It will be understood that the present invention has been described above by way of example only and that the above description should not be taken to impose any limitation on the scope of the claims. Specifically, although the present invention has been described with reference to a pMDI, the invention is not limited to this form of inhaler. The scope of the invention is defined by the appended claims.

The present application claims priority from UK patent application No. 05 187 70.3, filed 14 Sep. 2005, the entire content of which is hereby incorporated herein by reference.

What is claimed is:

1. A closure for use with a dispensing device which comprises a housing having a dispensing outlet, a supply of a substance and a dispensing member mountable in the housing in a rest position relative to the housing and movable from the rest position to an actuated position, said movement from the rest position to the actuated position causing dispensing of said substance out of the dispensing outlet, wherein the closure is adapted to close the dispensing outlet in two different orientations of the closure, and wherein the closure has a restricting member which, when the closure closes the dispensing outlet in either orientation, prevents movement of the dispensing member from the rest position to the actuated position so that dispensing of the substance is prevented when the closure closes the dispensing outlet.

2. A closure for use with a dispensing device which comprises a housing having a dispensing outlet, a supply of a substance and a dispensing member mountable in the housing in a rest position relative to the housing and movable from the rest position to an actuated position, movement from the rest position to the actuated position causing dispensing of said substance out of the dispensing outlet, wherein the closure is adapted to close the dispensing outlet in first and second orientations of the closure, wherein the closure has a restricting member to restrict movement of the dispensing member from the rest position towards the actuated position when the closure closes the dispensing outlet, such that dispensing of the substance is prevented, and wherein the restricting member is adapted to move between a first configuration on the closure, which is adoptable when the closure closes the dispensing outlet in the first orientation, and a second configuration on the closure, which is adoptable when the closure closes the dispensing outlet in the second orientation.

3. The closure of claim 1, wherein the restricting member is adapted to move between a first configuration on the closure, which is adoptable when the closure closes the dispensing outlet in the first orientation, and a second configuration on the closure, which is adoptable when the closure closes the dispensing outlet in the second orientation.

4. The closure of claim 2, wherein the restricting member is movably mounted on the closure to enable movement between the first and second configurations.

5. The closure of claim 4, wherein the restricting member is pivotally mounted on the closure.

6. The closure of claim 1, wherein the restricting member is adapted to pass through the dispensing outlet when the closure closes the dispensing outlet in either orientation thereof.

7. The closure of claim 1, wherein the restricting member is provided with a fastener for fastening the closure to the dispensing device when the closure closes the dispensing outlet in the first and second orientations.

8. The closure of claim 7 configured and arranged such that the fastener is able to fasten to the same feature of the dispensing device irrespective of the orientation of the closure when closing the dispensing outlet.

9. The closure of claim 7, wherein the fastener is a clip.

10. The closure of claim 7, wherein the fastener is located at a free end of the restricting member.

11. (canceled)
